(19) United States
(12) Patent Application Publication
(10) Pub. No.: US 2020/0093461 A1
(43) Pub. Date: Mar. 26, 2020

(54) Method and Apparatus for Using Doppler Guidance to Locate a Catheter Across a Stenotic Aortic Valve

(71) Applicant: Leon J. Frazin, Glencoe, IL (US)

(72) Inventor: Leon J. Frazin, Glencoe, IL (US)

(21) Appl. No.: 16/137,077

(22) Filed: Sep. 20, 2018

(51) Int. Cl. A61B 8/08; A61B 34/20; A61B 8/00; A61B 90/00

(52) U.S. Cl. CPC A61B 8/0841; A61B 34/20; A61B 8/06; A61B 8/445; A61B 90/39; A61B 8/488

(57) ABSTRACT

A method and apparatus for locating a Doppler ultrasound transceiver tipped guide wire in the orifice of a stenotic aortic valve. The guide wire is inserted into a peripheral blood vessel, and the transceiver generates electrical signals reflective of the maximum velocity of the blood passing through the stenotic aortic valve, thus aligning the tip of the guide wire with the axis of the orifice of the stenotic aortic valve. Upon removal of the guide wire, a catheter is inserted over the guide wire. The guide wire is removed from the catheter, and a second, stiffer guide wire is inserted through the catheter, and the catheter is removed. A sheath comprising a collapsed caged bioprosthetic valve circumscribing a dilation balloon is inserted over the second guide wire to a location in the orifice of the stenotic aortic valve, which has been located by blood velocity images shown on a display screen electrically connected to a velocimeter. Upon inflation of the dilation balloon, the caged bioprosthetic valve is moved into contact with the orifice of the stenotic aortic valve.

### BACKGROUND OF THE INVENTION

The present invention relates to an apparatus and method for accurately locating a guide wire in a stenotic aortic valve by detecting the maximum blood velocity through the stenotic aortic valve and locating the guide wire based on the determination of the maximum blood velocity. Specifically, the disclosed invention pertains to an apparatus and method for the accurate placement of a guide-wire mounted Doppler ultrasound transceiver into the left ventricle chamber of the heart as a diagnostic procedure, or to replace a damaged aortic valve using a transcatheter aortic valve replacement (TAVR) procedure.

Aortic stenosis is one of the most serious heart valve disease problems, according to the American Heart Association. Aortic stenosis is an abnormal narrowing or stricture in the aortic valve opening, which results in restricted blood flow from the heart's left ventricle to the aorta of the heart and then to the rest of the patient's body. This restriction affects the pressure dynamics in the left ventricle and can lead to disability and death. Aortic stenosis may result from a congenital heart defect (a bicuspid aortic valve), or from the aging process as calcium or scarring damages the aortic valve, causing a restriction in the valve opening.

In a stenotic aortic valve, the flaps of the valve thicken, stiffen or fuse together, such that the valve cannot fully open. This condition forces the heart to work harder to pump blood through the valve. Also, the body may suffer from a reduced supply of oxygen.

The aim of heart valve surgery is to cure the valve problem, improve cardiac function, and lengthen life by restoring the functioning of the heart valve. In occasional cases, where practical, it is preferable to repair a valve by retaining the patient's own heart tissue. Usually, however, replacement valve may be obtained from another human or animal, such as cow or pig heart tissue. A manufactured mechanical valve may also be used.

One stenotic aortic valve repair procedure is balloon valvuloplasty, which is part of a cardiac catheterization procedure aimed at relieving valve tightness. This procedure is less invasive then open-heart surgery. Balloon valvuloplasty comprises threading a catheter mounting an expandable balloon into the tightened aortic valve, and expanding the balloon to stretch the valve open and separate the valve's leaflets. The balloon and catheter are then removed, and in a successful procedure, the aortic valve will move to the open position as required, but usually causes valve leakage.

Where the medical staff treating a patient with a stenotic aortic valve concludes that a balloon valvuloplasty or similar valve repair procedure is contraindicated, the patient's stenotic valve is replaced. TAVR is a recent stenotic valve replacement procedure, which is initiated by advancing a catheter into the femoral or brachial artery, or other blood vessel leading to the heart, until the tip of the catheter is positioned in the strictured opening of the aortic valve. Next, a fully collapsed cage-mounted bioprosthetic valve, and a collapsed balloon inside the collapsed bioprosthetic valve, are inserted into a lumen of the catheter, and the cage-mounted bioprosthetic valve and balloon are advanced to the opening of the aortic valve. Once in this position, the cage mounted bioprosthetic valve is expanded by inflating the balloon, such that the new bioprosthetic valve is wedged into place at the wall of the aortic valve. Once the new aortic valve is securely in place, the catheter and balloon are removed.

In both the stenotic aortic valve repair and replacement procedures described above, the tip of the catheter must be carefully guided through the blood vessel of the patient so that the tip reaches the narrow opening of the aortic valve in the left ventricular chamber of the heart. The method used presently to guide the catheter to its proper position uses a non-Doppler guided guide wire to initially cross the aortic valve into the left ventricular chamber. Next, a catheter is advanced over the guide wire and the tip of the catheter is advanced into the left ventricular chamber. The guide wire is then removed from the catheter, and replaced with a stiffer guide wire. The stiffer guide wire remains in the left ventricular chamber, and the TAVR apparatus is advanced over the stiffer guide wire. The catheter is removed, and the TAVR procedure is administered. A disadvantage of present X-ray guidance is the length of time it takes to advance a catheter into the small opening of a stenotic aortic valve using this method, where the patient and operators are subject to radiation exposure during this time period. Another problem using an X-ray guided method is that the tip of the guide wire or catheter may impact or poke the calcified flaps of the aortic valve, with a force sufficient to chip off portions of the valve. If these calcified chips enter the blood stream, a stroke or other systemic emboli may occur.

Applicant's U.S. Pat. No. 5,220,924 (the “'924 patent”) discloses a method and apparatus for Doppler-guided intravascular catheterization by inserting into a peripheral blood vessel a steerable catheter having a Doppler ultrasound transceiver at its tips. The Doppler crystal of the '924 patent detects the presence of antegrade or retrograde blood flow across the crystal. However, the transceiver of the '924 patent is not described as measuring blood velocity in a stenotic aortic valve. The flow detection of the '924 patent method is a function of the direction of blood flow to or away from the Doppler crystal, not blood velocity. In the method and apparatus of the present invention, a Doppler crystal generates a signal responsive to the velocity of blood across the aortic valve. Merely detecting the direction of blood flow as the Doppler crystal is guided in the vicinity of a stenotic aortic valve will not enable the user to place the catheter tip precisely in the aortic valve narrowed orifice.

### BRIEF DESCRIPTION OF THE DRAWINGS

An embodiment of the presently disclosed invention is illustrated with reference to the following drawings in which:

FIG. 1 is a schematic illustration of the human body and heart of a human body, showing the femoral arteries and the brachial arteries leading to and connecting with the aorta of the heart;

FIG. 2 is a cut-away schematic view of a human heart, showing the location of a stenotic aortic valve;

FIG. 3 illustrates a healthy aortic valve in an open and closed position;

FIG. 4 illustrates a stenotic aortic valve in a partially opened and closed position;

FIG. 5 is a partial cut-away view of a human heart, with a cage-mounted collapsed bioprosthetic replacement valve surrounding a collapsed dilation balloon inserted into a stenotic aortic valve annulus;

FIG. 6 is a partial cut-away view of a human heart, showing the installation of a cage-mounted bioprosthetic replacement valve wedged into the aortic valve annulus through expansion of a dilation balloon; and

FIG. 7 is a schematic view of a guide wire extending through a catheter, where the guide wire is electrically connected to an external power source and a display monitor.

### SUMMARY OF THE INVENTION

A method and apparatus for advancing the tip of a guide wire through the orifice of a stenotic aortic valve into the left ventricle chamber of the heart. A catheter is initially inserted into a peripheral blood vessel leading to a patient's heart. A straight-tipped guide wire having a Doppler blood flow direction and blood flow velocity ultrasound transceiver at the tip of the guide wire is inserted into the catheter, and the ultrasound transceiver is advanced through the blood vessel until the tip of the guide wire approaches the stenotic aortic valve that admits blood from the left ventricular chamber of the heart into the aorta. A signal is generated by the ultrasound transceiver located on the tip of the guide wire reflective of the velocity of the blood passing through the stenotic aortic valve, and this signal is received by a velocimeter which ultimately displays the blood velocity signal on a visual display device. The tip of the Doppler guide wire is guided to a location where the maximum blood flow velocity through the stenotic aortic valve is determined. Once the guide wire is at a position where maximum blood velocity is detected, the tip of the Doppler guide wire is advanced through the stenotic aortic valve orifice and into the left ventricular chamber.

Next, the catheter is advanced over the guide wire until the tip of the catheter is in the left ventricular chamber. The Doppler tipped guide wire is then removed from the catheter, and a second, stiffer guide wire is inserted into the catheter, with the tip of the stiffer guide wire entering the left ventricular chamber. The catheter is then removed while leaving the stiffer guide wire in place. A sheath is then inserted over the stiffer guide wire, which sheath includes a collapsed dilation balloon and a collapsed caged bioprosthetic valve assembly. The collapsed balloon and caged valve assembly is advanced over the stiffer guide wire until the assembly is adjacent the stenotic aortic valve orifice. The balloon and the caged valve are then inflated to expand and place the caged valve into contact with the inner surface tissue of the aortic valve. The dilation balloon is then deflated, and removed from the peripheral blood vessel while the expanded bioprosthetic valve remains in place in the stenotic aortic valve orifice. As a final step, the stiffer guide wire is removed from the peripheral blood vessel.

### DETAILED DESCRIPTION OF THE ILLUSTRATED EMBODIMENT

The invention is described and claimed primarily in connection with the catheterization of a stenotic aortic valve orifice of a human or animal heart, however other procedures are likewise possible using the Doppler guidance and blood velocity detection system of the method and apparatus described below.

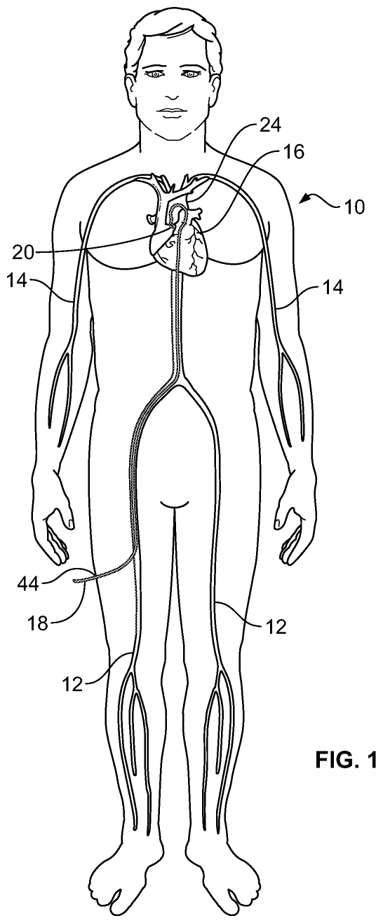

FIG. 1 illustrates a human body 10 and the peripheral femoral arteries 12 and brachial arteries 14 leading to the aorta 24 of heart 16. A Doppler crystal tipped guide wire 18 is shown inserted into a femoral artery 12, and the tip of guide wire 18 has been advanced through femoral artery 12 to aorta 24 and into the vicinity of the aortic valve 20 of heart 16. For purposes of this description, aortic valve 20 is damaged, or stenotic, and requires repair or replacement.

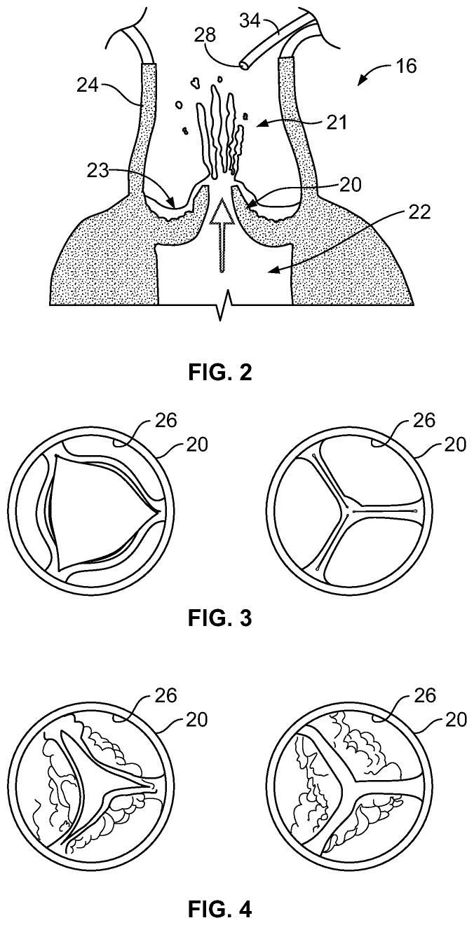

FIG. 2 is a detailed illustration of blood when pumped from the left ventricle 22 of heart 16 through a stenotic aortic valve 20 and into the aorta 24. From aorta 24, blood flows to the rest of the body 10. As the aortic valve 20 becomes stenotic through disease or age, the opening in the aortic valve 20 becomes narrow and obstructs the opening of the valve, making it more difficult for the heart 16 to pump blood into the aorta. In FIG. 2, the maximum velocity of blood passing through aortic valve 20 is indicated by the numeral 21, and a lower blood velocity is indicated by the numeral 23.

FIG. 3 illustrates a healthy tricuspid aortic valve 20 in both its open and closed positions. The heart muscle regulates the opening and closing of aortic valve 20 in coordination with the pumping action of heart 16, such that blood passes freely through valve 20 when the valve is in its open position. FIG. 4 illustrates the partially open and closed position of a stenotic aortic valve 20, where due to the damage to valve 20, the valve is only partially open when it should be substantially fully open, and may not fully close were the valve 20 healthy.

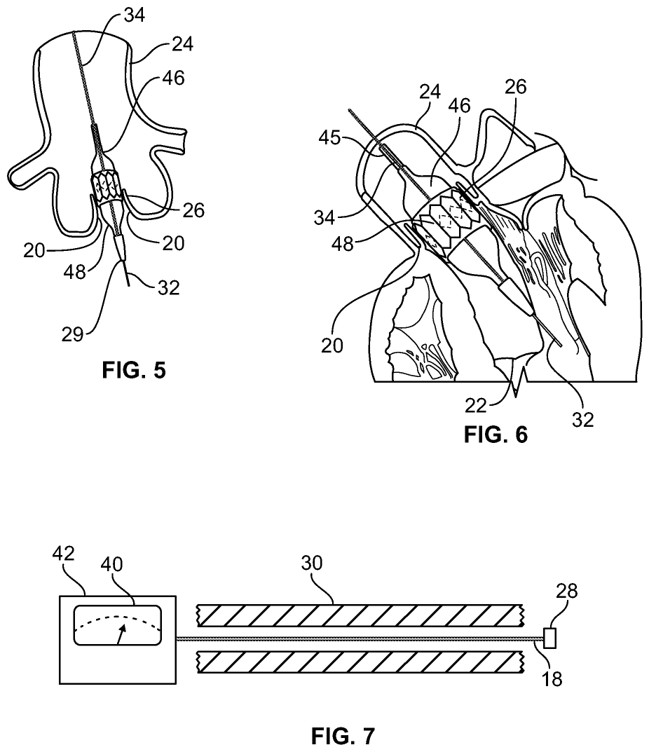

As stated above, medical procedures have been developed over the last several years to allow replacement or repair of a stenotic aortic valve 20, such as the TAVR procedure. These medical procedures are predicated upon advancing and guiding the tip of a catheter to the orifice or opening 26 (FIGS. 5, 6) of stenotic aortic valve 20. Once the catheter tip is properly located in the orifice 26 of the stenotic aortic valve, a dilation balloon is inflated and a caged bioprosthetic valve is expanded into the orifice 26, as shown in FIGS. 5 and 6 and as will be explained.

Where cardiac catheterization is used as a diagnostic procedure or to repair or replace a stenotic aortic valve 20, the Doppler tipped 28 guide wire 18 is inserted into the femoral artery 12 or brachial artery 14 using a suitable insertion sheath (not shown). Guide wire 18 is advanced in the artery against the direction of blood flow. The guide wire 18 is advanced until it reaches the vicinity of the stenotic aortic valve 20 (FIGS. 1 and 2). At this point, Doppler ultrasound transceiver crystal 28 generates an electrical signal reflective of the velocity of the blood at the transceiver crystal 28. Guide wire 18 is manipulated by the user so that the transceiver crystal 28 obtains blood velocity signals from the vicinity of the aortic valve 20. Due to the restrictive qualities of the aortic valve 20 applied to the movement of blood through aortic valve orifice 26, the maximum blood velocity 21 (FIG. 2) will be through the aortic valve orifice 26 itself. Therefore, once the transceiver crystal 28 is located at the point of highest blood velocity 21 as displayed on the blood velocity display 40 of the monitor of an external device 42 (FIG. 7), the user knows that tip of guide wire 18 is located in the orifice 26 of stenotic aortic valve 20. The guide wire 18 and the transceiver crystal 28 are advanced until the guide wire 18 has crossed the stenotic aortic valve 20 into the left ventricular chamber 22.

Once the tip of the Doppler guide wire 18 has crossed through stenotic aortic valve 20, the catheter 30 (FIG. 7) which has supported the guide wire is placed over guide wire 18. Next, catheter 30 is advanced over the guide wire 18 until the tip of catheter 30 is in the left ventricular chamber 22. The Doppler tipped guide wire 18 is then removed from catheter 30 and a second, stiffer guide wire 32 is inserted into the catheter 30, with the tip of the stiffer guide wire 32 entering the left ventricular chamber 22. The catheter 30 is then removed while leaving the stiffer guide wire 32 in place. A sheath 34 is then inserted over the stiffer guide wire 32 which sheath 34 includes a collapsed dilation balloon 36 and a collapsed caged bioprosthetic valve 38. The collapsed balloon 36 and caged valve 38 are advanced over the stiffer guide wire 32 until the collapsed balloon 36 and caged valve 38 are adjacent the stenotic aortic valve orifice 26. The balloon 36 is then inflated to expand and place the caged valve 38 into contact with the inner surface tissue of the aortic valve 20. The dilation balloon 36 is then deflated, and removed from the peripheral blood vessel while the expanded bioprosthetic valve 38 remains in place in the stenotic aortic valve orifice 26. As a final step, the stiffer guide wire 32 is removed from the peripheral blood vessel.

An external power source 42 (FIG. 7) including a display screen 40, serves as a power supply for Doppler transceiver crystal 28 as the transceiver generates an ultrasound carrier frequency of approximately 20 megahertz. The external power source 42 also displays separate images on a monitor 40 representative of both blood velocity and blood flow direction at transceiver crystal 38 in the corresponding artery 12, 14.

X-ray fluoroscopy may be used to guide guide wire 18 and to confirm that guide wire 18 has been properly located in the aortic root and in the left ventricular chamber. In the above-described illustration of an embodiment of the present invention, Doppler crystal 28 does not measure blood pressure.

The presently disclosed apparatus could also be used in a diagnostic procedure to check the tightness of an aortic valve to determine if the valve is working properly.

While the present disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

I claim:

1. A method for locating the tip of a guide wire in an orifice of a stenotic aortic valve, a Doppler ultrasound transceiver mounted on the tip of the guide wire, comprising the steps of:
inserting the guidewire into a peripheral blood vessel;
guiding the tip of the guide wire through an aorta to the vicinity of the stenotic aortic valve;
obtaining a signal from the ultrasound transceiver reflective of the velocity of blood passing through the stenotic aortic valve;
guiding the tip of the guide wire to a location where the maximum velocity of blood passing through the stenotic aortic valve is determined;
advancing the tip of the guide wire through the orifice of the stenotic aortic valve at the location where the maximum velocity of blood passing through the stenotic aortic valve is determined.

2. The method of claim 1, wherein:
the step of guiding the tip of the guide wire to a location where the maximum velocity of blood passing through the stenotic aortic valve is determined comprises the step of manipulating the guidewire.

3. The method of claim 1, wherein:
the step of guiding the tip of the guide wire through the peripheral blood vessel comprises the step of generating from signals produced by the ultrasound transceiver a continuous indication of the direction of blood flow relative to the tip of the guide wire.

4. The method of claim 1, including the additional step of:
advancing the guidewire across the orifice of the stenotic aortic valve and into a left ventricle of the heart.

5. The method of claim 1, including the further steps of:
advancing a first catheter over the guide wire to place the tip of the catheter into a left ventricular chamber of the heart;
removing the guide wire from the catheter;
advancing a second guide wire through the first catheter;
removing the catheter from the second guide wire;
advancing a sheath having a collapsed dilation balloon and a collapsed caged bioprosthetic valve over the second guide wire in a direction toward the stenotic aortic valve;
positioning the collapsed caged bioprosthetic valve and collapsed dilation balloon in the orifice of the stenotic aortic valve;
inflating the dilation balloon to expand the caged bioprosthetic valve into contact with inner surfaces of the stenotic aortic valve;
deflating the dilation balloon;
removing the sheath and the dilation balloon from the peripheral blood vessel; and
removing the guide wire from the peripheral blood vessel.

6. The method of claim 1, comprising the further step of:
confirming that the guide wire tip is properly located in the orifice of the stenotic aortic valve by fluoroscopy.

7. The method of claim 5, wherein:
the second guide wire is stiffer than the first guide wire.

8. An apparatus for locating the tip of a guide wire in the orifice of a stenotic aortic valve, comprising:
a Doppler ultrasound transceiver mounted on the tip of the guide wire;
the Doppler ultrasound transceiver adapted to generate a first electrical signal reflective of the velocity of a fluid passing adjacent the Doppler ultrasound transceiver;
at least one electrical connector extending from said Doppler ultrasound transceiver to a velocimeter, said at least one electrical connector adapted to transmit the electrical signal generated by the Doppler ultrasound transducer to the velocimeter;
the velocimeter electrically connected to a display device, the velocimeter generating a second electrical signal, said second electrical signal creating an image on the display device;
the image indicating when the Doppler ultrasound transceiver is located at a point of maximum blood velocity through the stenotic aortic valve.
